(19) United States
(12) Patent Application Publication
(10) Pub. No.: US 2005/0283138 A1
(43) Pub. Date: Dec. 22, 2005

(54) Operating room control system

(75) Inventors: Koichi Tashiro, Sagamihara-shi (JP); Takeaki Nakamura, Tokyo (JP); Akinobu Uchikubo, Iruma-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, TOKYO (JP)

(21) Appl. No.: 11/157,445

(22) Filed: Jun. 21, 2005

(30) Foreign Application Priority Data
Jun. 21, 2004 (JP) 2004-182134

(51) Int. Cl. A61B 17/00

(52) U.S. Cl. 606/1

(57) ABSTRACT

An operating room control system including a medical device control system for controlling one or more medical devices for conducting a surgical procedure and a medical support device control system for controlling one or more medical support devices, which support the surgical procedure, wherein the medical support devices can be controlled from the side of the medical devices. For example, the operating environment of an operating section of the medical device control system is changed to enable a command input to the medical support device control system. Thereby, the medical support devices can be controlled from the side of the medical system. If the operating section is a touch panel having a display function, it is preferable to switch between the display function and input sensing function in synchronization with each other.

### CROSS-REFERENCE TO RELATED APPLICATION

This application is based upon and claims the benefit of priority from prior Japanese Patent Application No. 2004-182134, filed Jun. 21, 2004, the entire contents of which is incorporated herein by reference.

### BACKGROUND OF THE INVENTION

1. Field of the Invention

The present invention relates to an operating room control system comprising a medical system and an AV system.

2. Description of the Related Art

In recent years, an endoscopic surgery system for conducting surgical procedures using an endoscope has come into general use and a large variety of medical devices are in practical use.

The medical devices for use in this type of endoscopic surgery system include an electric cautery, an ultrasonic device, and an insufflator in addition to an electronic endoscopic system. For example, as suggested in the Laid-Open Japanese Patent Publication No. 2003-76786 or 2003-70746, the endoscopic operation system integrally manages these devices as a system and operating devices disposed under a system controller control these devices.

On the other hand, various peripheral devices are also placed besides the medical devices in the operating room where there is placed a medical system typified by the endoscopic surgery system. These peripheral devices are, for example, a server for storing reference images such as patient's pre-surgery CT images or ultrasound images referenced for surgical procedures, a display device for displaying the reference images, and an output device for recording or printing endoscopic images picked up by the endoscopic surgery system. Moreover, a teleconference system for communication with the outside of the operating room and a room light for controlling lighting of the operating room are placed as peripheral devices.

Due to the presence of these peripheral devices in various forms, there is constructed an AV system, which is a nonmedical device system separate from the system for controlling the medical devices, and it is controlled by operating devices other than the operating devices for controlling the medical devices.

### BRIEF SUMMARY OF THE INVENTION

According to one aspect of the present invention, there is provided an operating room control system comprising a medical device control system for controlling one or more medical devices for conducting a surgical procedure and a medical support device control system for controlling one or more medical support devices, which support the surgical procedure, wherein a command input to the medical support device control system is enabled by changing an operating environment of an operating section of the medical device control system.

Thereby, the one or more medical support devices can be controlled from the side of the medical system.

For example, if the operating section comprises a touch panel, an operator can switch between a display function and a sensing function of the touch panel of the operating section in synchronization with each other when changing the operating environment.

Thereupon, the operating environment changes between the display function and the input function in synchronization with each other, whereby operating errors can be minimized. In this instance, it is also possible to have a configuration in which the display function and the sensing function are transmitted via separate signal paths and are changed in different switching methods.

If the medical support device control system also has an operating section (a second operating section), the operating environment can be changed by switching between the operating environment of the operating section and an operating environment of the second operating section.

### BRIEF DESCRIPTION OF THE SEVERAL VIEWS OF THE DRAWINGS

These and other features, aspects, and advantages of the apparatus and methods of the present invention will become better understood with regard to the following description, appended claims, and accompanying drawings where:

FIG. 1 is a schematic diagram showing a configuration of an endoscopic surgery system according to a first embodiment of the present invention;

FIG. 2 is a block diagram showing a configuration of an operating room control system having the endoscopic surgery system shown in FIG. 1;

FIG. 3 is a block diagram showing a configuration of a relevant part in FIG. 2;

FIG. 4 is a first diagram illustrating an action of the relevant part shown in FIG. 3;

FIG. 5 is a second diagram illustrating an action of the relevant part shown in FIG. 3;

FIG. 6 is a block diagram showing a configuration of an operating room control system according to a second embodiment of the present invention;

FIG. 7 is a block diagram showing a configuration of a relevant part in FIG. 6;

FIG. 8 is a first diagram illustrating an action of the relevant part shown in FIG. 7;

FIG. 9 is a second diagram illustrating an action of the relevant part shown in FIG. 7;

FIG. 10 is a block diagram showing a configuration of a relevant part of an operating room control system according to a third embodiment of the present invention;

FIG. 11 is a first diagram illustrating an action of the relevant part shown in FIG. 10; and

FIG. 12 is a second diagram illustrating an action of the relevant part shown in FIG. 10.

### DETAILED DESCRIPTION OF THE PREFERRED EMBODIMENTS

Preferred embodiments of the invention are described below with reference to the accompanying drawings.

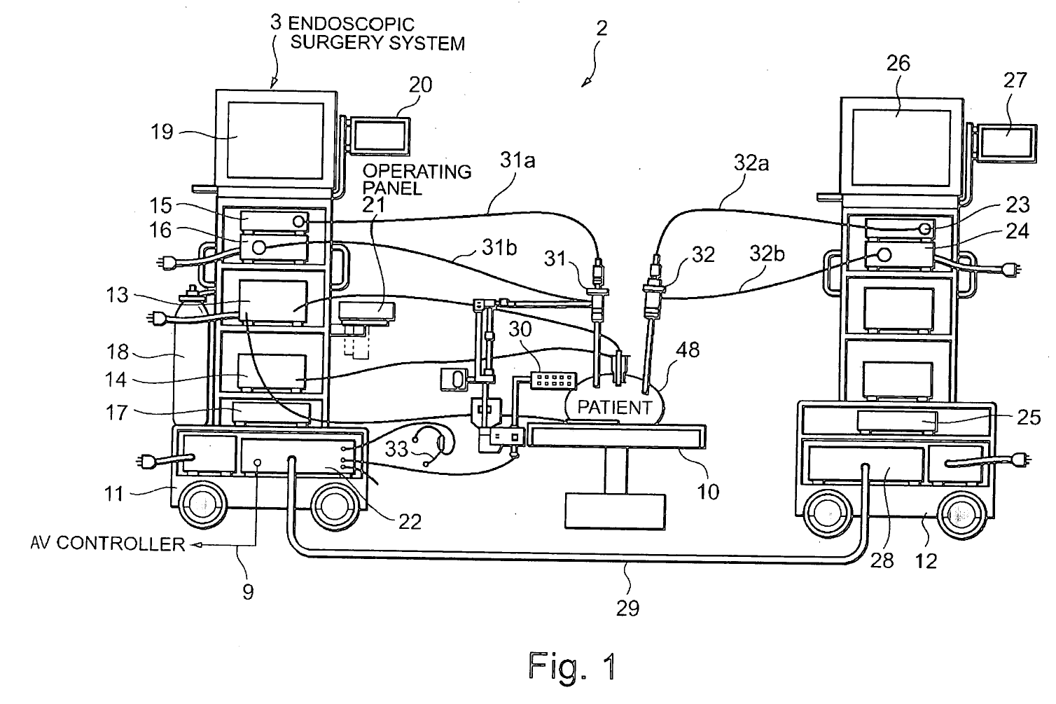

First, a configuration of an endoscopic surgery system 3 placed in an operating room 2 will be described hereinafter by reference to FIG. 1.

As shown in FIG. 1, a patient's bed 10 on which a patient 48 is laying and the endoscopic surgery system 3 are placed in the operating room 2. The endoscopic surgery system 3 has a first cart 11 and a second cart 12.

The first cart 11 is loaded with medical devices as controlled devices such as, for example, an electric cautery 13, an insufflator 14, an endoscopic camera 15, a light source 16, a video tape recorder (VTR) 17, and other devices and a gas cylinder 18 filled with carbon dioxide. The endoscopic camera 15 is connected to a first endoscope 31 via a camera cable 31a. The light source 16 is connected to the first endoscope 31 via a light guide cable 31b.

Moreover, the first cart 11 is loaded with a display device 19, a first integrated display panel 20, an operating panel 21, and the like. The display device 19 is, for example, a TV monitor for displaying endoscopic images or the like.

The integrated display panel 20 is a display member capable of selectively displaying various data during surgery. The operating panel 21 comprises a display section such as, for example, a liquid crystal display, and a touch sensor integrally arranged on the display section, for example. The operating panel 21 is an integrated operating device, which is operated by a nurse or the like in a nonsterilized area.

Furthermore, the first cart 11 is loaded with a system controller 22, which is a medical device controller for controlling medical devices. The system controller 22 is connected to the electric cautery 13, the insufflator 14, the endoscopic camera 15, the light source 16, and the VTR stated above via communication lines (not shown). The system controller 22 can be connected to a headset-type microphone 33. Thereby, the system controller 22 can recognize voice input from the microphone 33 and can control devices according to a surgeon's voice.

On the other hand, the second cart 12 is loaded with an endoscopic camera 23, a light source 24, an image processing device 25, a display device 26, and a second integrated display panel 27, which are controlled devices.

The endoscopic camera 23 is connected to a second endoscope 32 via a camera cable 32a. The light source 24 is connected to the second endoscope 32 via a light guide cable 32b.

The display device 26 displays endoscopic images picked up by the endoscopic camera 23. The second integrated display panel 27 can selectively display various data during surgery.

The endoscopic camera 23, the light source 24, and the image processing device 25 are connected to a repeater unit 28 mounted on the second cart 12 via communication lines (not shown). The repeater unit 28 is connected to the system controller 22 mounted on the first cart 11 via a trunk cable 29.

The system controller 22 can integrally control the camera 23, the light source 24, and the image processing device 25 mounted on the second cart 12 and the electric cautery 13, the insufflator 14, the camera 15, the light source 16, and the VTR 17 mounted on the first cart 11. If the system controller 22 is communicating with these devices, the system controller 22 can display the setting conditions of the connected devices and the setting screens of control switches on the liquid crystal display of the operating panel 21. Moreover, the system controller 22 can enter an operation such as a change of a setting value through an operation on the touch panel in a given area in response to a touch on the setting screen of a desired control switch of the operating panel 21.

A remote controller 30 is a second integrated operating device operated by a surgeon or the like in the sterilized area. It can control other devices, for which the communication is established, via the system controller 22.

The system controller 22 is connected to a patient monitor system 4 via a cable 9. As described later, the system controller 22 can analyze biological information acquired from the patient monitor system 4 and can display the analysis result on a required display device.

Moreover, the system controller 22 is equipped with an infrared communication port (not shown), which is a communication member. The infrared communication port is provided in a position where it is readily irradiated with infrared rays such as, for example, in the vicinity of the display device 19 and is connected to the system controller 22 via a cable.

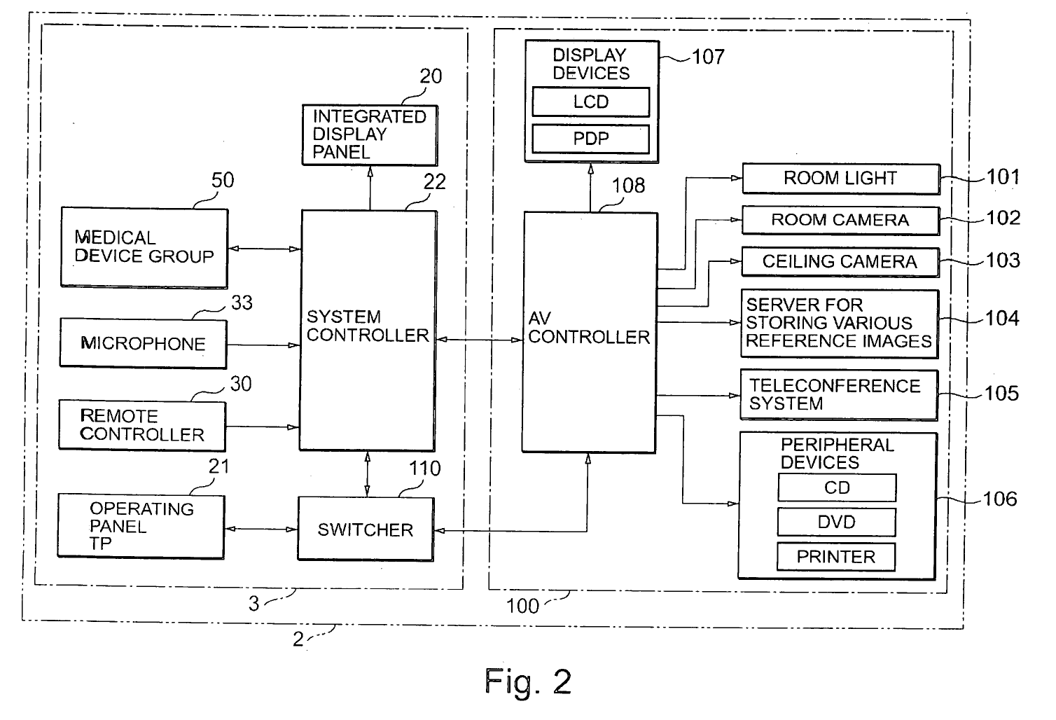

As shown in FIG. 2, there is constructed an operating room control system with an endoscopic surgery system 3 in an operating room 2. In the operating room control system, an AV system 100 is arranged besides the endoscopic surgery system 3. The endoscopic surgery system 3 comprises an integrated display panel 20, an operating panel 21, a system controller 22, a microphone 33, a remote controller 30, and a medical device group 50 including a light source 24. The AV system 100 comprises various audio-visual devices or lighting devices, which are nonmedical devices. These devices are medical support devices for supporting medical treatment.

More specifically, the AV system 100 includes a room light 101, a room camera 102, a ceiling camera 103, a server for storing various reference images 104, a teleconference system 105, various peripheral devices 106, various display devices 107, and an AV controller 108 as a medical support device control system for controlling such devices.

The server for storing various reference images 104 stores pre-surgery CT images or ultrasound images of a patient 48. These images can be displayed on an LCD or a PDP in the display devices 107.

The AV controller 108 is capable of transmitting or receiving information to or from the system controller 22 of the endoscopic surgery system 3 via a signal cable 9. Thereby, it can display an endoscopic image from the endoscopic surgery system 3 on the display devices 107 or output it to a CD, a DVD, and a printer as the peripheral devices 106.

The operating panel 21 of the endoscopic surgery system 3 is an operating section that can be used for displaying various information and entering commands related to the system controller 22 via a switcher 110. Moreover, the operating panel 21 can be used for displaying various information and entering commands related to the AV controller 108 via the switcher 110.

Subsequently, the switcher 110 as an operating environment changing section will be described in detail.

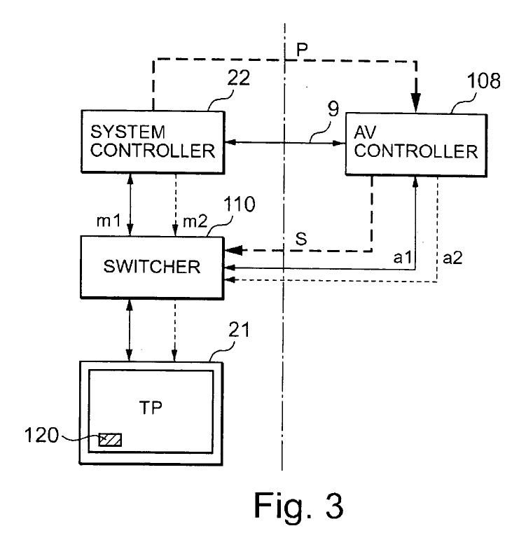

As shown in FIG. 3, to perform a touch panel function of the operating panel 21, the switcher 110 receives inputs of a serial signal m1 (for example, an RS-232C signal) as a control signal from the system controller 22, a VGA or other video signal m2, a serial signal al (for example, an RS-232C signal) as a control signal from the AV controller 108, and a VGA or other video signal a2.

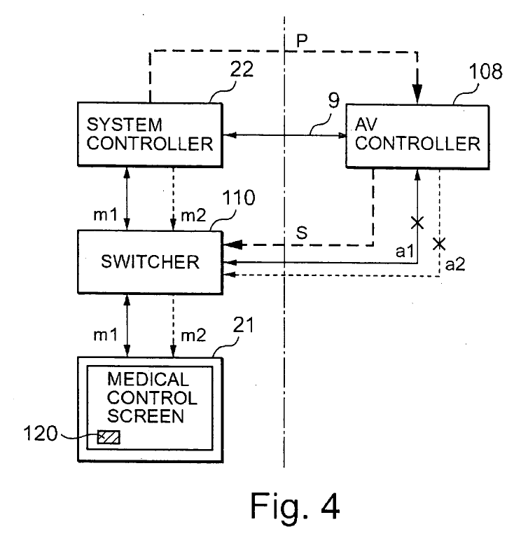

As shown in FIG. 4, the switcher 110 in an initial state is cut off from the serial signal al and the VGA or other video signal a2 as control signals from the AV controller 108. On the other hand, the switcher 110 outputs the serial signal m1 and the VGA or other video signal m2 as control signals from the system controller 22 to the operating panel 21 and displays a medical control screen on the operating panel 21, whereby the endoscopic surgery system 3 can be controlled.

If an operator selects a switching button 120 displayed on the operating panel 21 using the touch panel function under the control of the endoscopic surgery system 3, the selection signal is transmitted to the system controller 22 by means of a serial signal m1 and the system controller 22 transmits the information to the AV controller 108 by means of a parallel signal P. For example, the parallel signal P is a contact output signal.

Upon receiving the parallel signal P based on the switching button 120, the AV controller 108 outputs a control signal S for switching of the switcher.

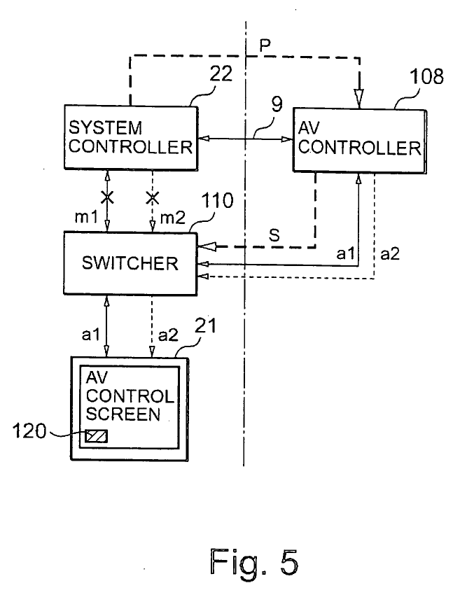

As shown in FIG. 5, the switcher then 110 turns off the serial signal m1 and the VGA or other video signal m2 as control signals from the system controller 22 by the control signal S and outputs the serial signal a1 and the VGA or other video signal a2 as control signals from the AV controller 108 to the operating panel 21 to display an AV control screen on the operating panel 21. This enables a control of the AV system 100 using the operating panel 21.

If the operator selects the switching button 120 displayed on the operating panel 21 using the touch panel function under the control of the AV system 100, the selection signal is transmitted to the AV controller 108 by means of a serial signal a1. Thereafter, the AV controller 108 outputs a control signal S for switching of the switcher 110, whereby the control state shown in FIG. 4 is resumed.

In this manner, the endoscopic surgery system 3 and the AV system 100 can be controlled by using the operating panel 21 arranged in the endoscopic surgery system 3 via the switcher 110 in this embodiment. This allows the system in a sterilized area (the endoscopic surgery system 3) to control the system in a nonsterilized area (the AV system 100) by using the single operating panel.

In other words, in this embodiment, the switcher 110 enables switching of the control environment on the operating panel 21, whereby the AV system can be easily controlled from the side of the medical system at a low cost.

A second embodiment is substantially the same as the first embodiment. Therefore, only different points will be described below. The same reference numerals are used for the identical parts and their description is omitted here.

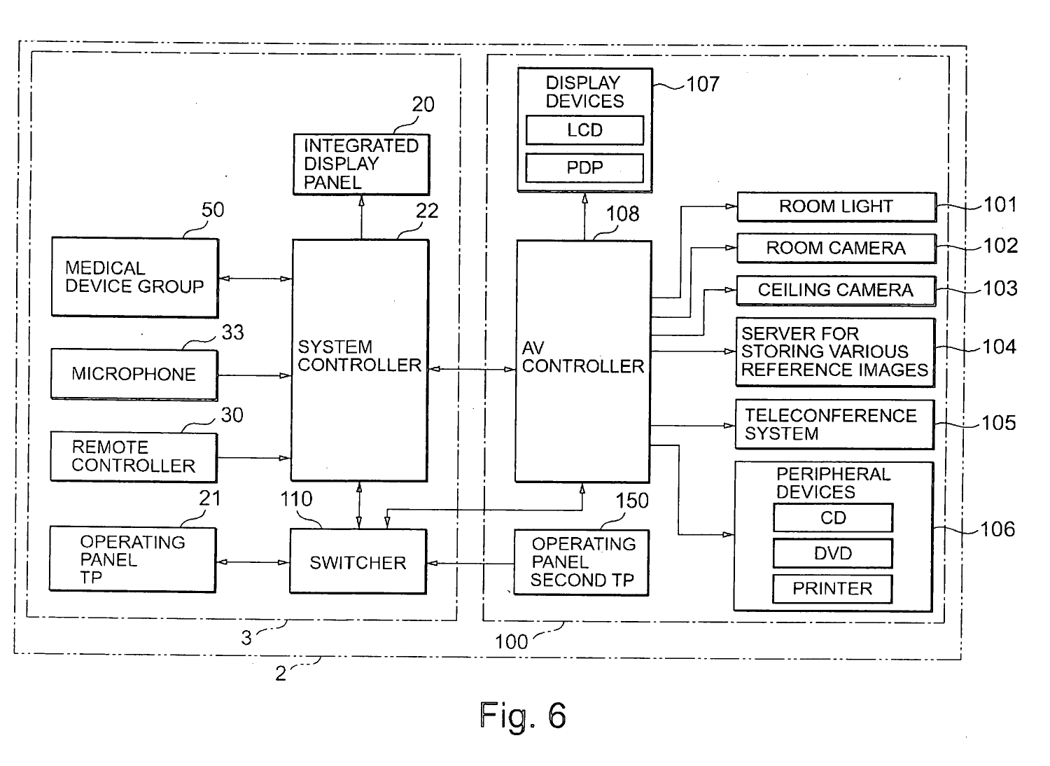

As shown in FIG. 6, the AV system 100 is also provided with an operating panel 150 having a touch panel function in this embodiment, with the operating panel 21 of the endoscopic surgery system 3 and the operating panel 150 of the AV system 100 connected to the switcher 110.

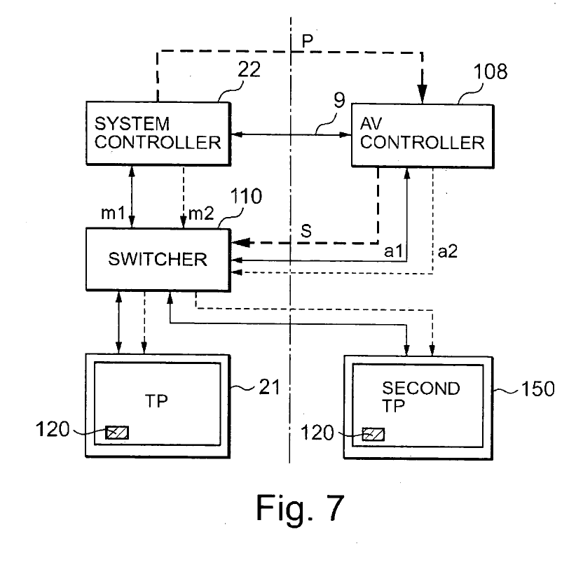

In other words, as shown in FIG. 7, the switcher 110 receives inputs of a serial signal m1 (for example., an RS-232C signal) and a VGA or other video signal m2 as control signals from the system controller 22 and a serial signal a1 (for example, an RS-232C signal) and a VGA or other video signal a2 as control signals from the AV controller 108 in order to perform the touch panel function of the operating panel 21. The switcher 110 then outputs the serial signal m1 and the video signal m2 or the serial signal a1 and the video signal a2 selectively to the operating panel 21 and the operating panel 150.

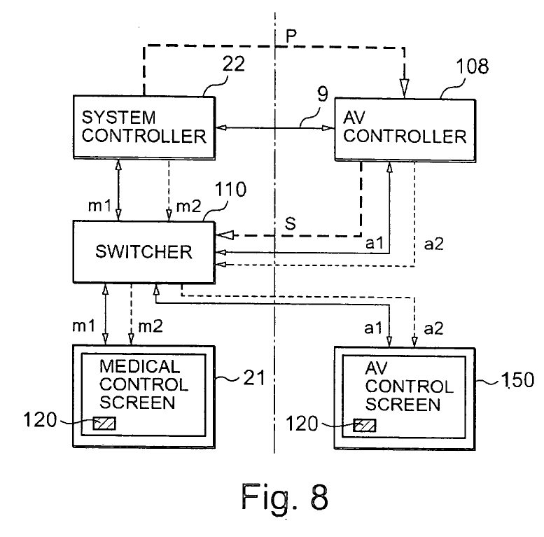

More specifically, as shown in FIG. 8, the switcher 110 outputs the serial signal ml and the video signal m2 from the system controller 22 to the operating panel 21 and outputs the serial signal a1 and the video signal a2 from the AV controller 108 to the operating panel 150 in the initial state. This allows the switcher 110 to make the endoscopic surgery system 3 controllable on the operating panel 21 by displaying a medical control screen on the operating panel 21 and to make the AV system 100 controllable on the operating panel 150 by displaying an AV control screen on the operating panel 150.

If an operator selects a switching button 120 displayed on the operating panel 21 using the touch panel function under the control of the endoscopic surgery system 3 with the operating panel 21, the selection signal is transmitted to the system controller 22 by means of a serial signal m1. The system controller 22 then transmits the information to the AV controller 108 by means of a parallel signal P.

Upon receiving the switching signal information, the AV controller 108 outputs a control signal S for switching of the switcher 110.

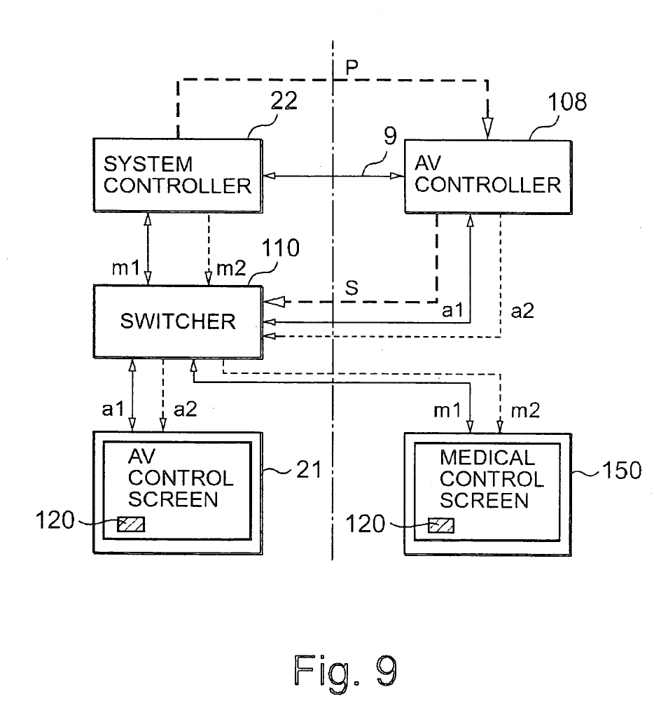

As shown in FIG. 9, by the control signal S, the switcher 110 outputs the serial signal m1 and the video signal m2 from the system controller 22 to the operating panel 150 and outputs the serial signal a1 and the video signal a2 from the AV controller 108 to the operating panel 21. This allows the switcher 110 to make the endoscopic surgery system 3 controllable on the operating panel 150 by displaying a medical control screen on the operating panel 150 and to make the AV system 100 controllable on the operating panel 21 by displaying an AV control screen on the operating panel 21.

If the operator selects the switching button 120 displayed on the operating panel 21 using the touch panel function also under the control of the AV system 100 with the operating panel 21, the selection signal is transmitted to the AV controller 108 by means of the serial signal a1. Then, the AV controller 108 outputs a control signal S for switching of the switcher 110, whereby the control state shown in FIG. 8 is resumed.

In this manner, similar effects as in the first embodiment can be achieved with the second embodiment. Therefore, the system in a sterilized area (the endoscopic surgery system 3) can control the system in a nonsterilized area (the AV system 100) by using the operating panel of the system in the sterilized area (the endoscopic surgery system 3).

A third embodiment is substantially the same as the second embodiment. Therefore, only different points will be described below. The same reference numerals are used for the identical parts and their description is omitted here.

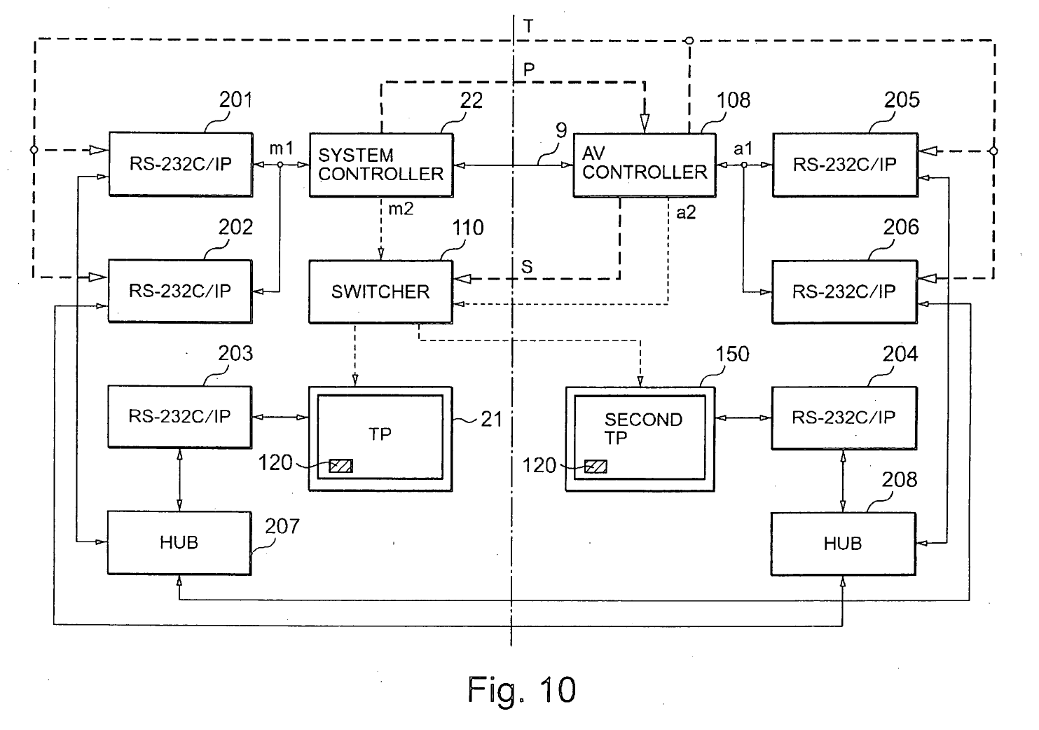

In this embodiment, as shown in FIG. 10, the switcher 110 makes switching of only video signals m2 and a2, while six serial/network protocol converters 201-206 (hereinafter, each of the serial/network protocol converters are referred to as an RS-232C/IP) and two hubs 207 and 208 perform switching controls of serial signals m1 and a1.

In other words, the serial signal m1 from the system controller 22 is entered into two RS-232C/IPs 201 and 202 and converted to network protocol signals. Output from the RS-232C/IP 201 is entered into the hub 207 and output from the RS-232C/IP 202 is entered into the hub 208. A network protocol signal from the hub 207 is converted to a serial communication via the RS-232C/IP 203 and output to the operating panel 21. A network protocol signal from the hub 208 is converted to a serial communication via the RS-232C/IP 204 and output to the operating panel 150.

Similarly, the serial signal a1 from the AV controller 108 is entered into two RS-232C/IPs 205 and 206 and converted to network protocol signals. An output from the RS-232C/IP 205 is entered into the hub 208 and an output from the RS-232C/IP 206 is entered into the hub 207.

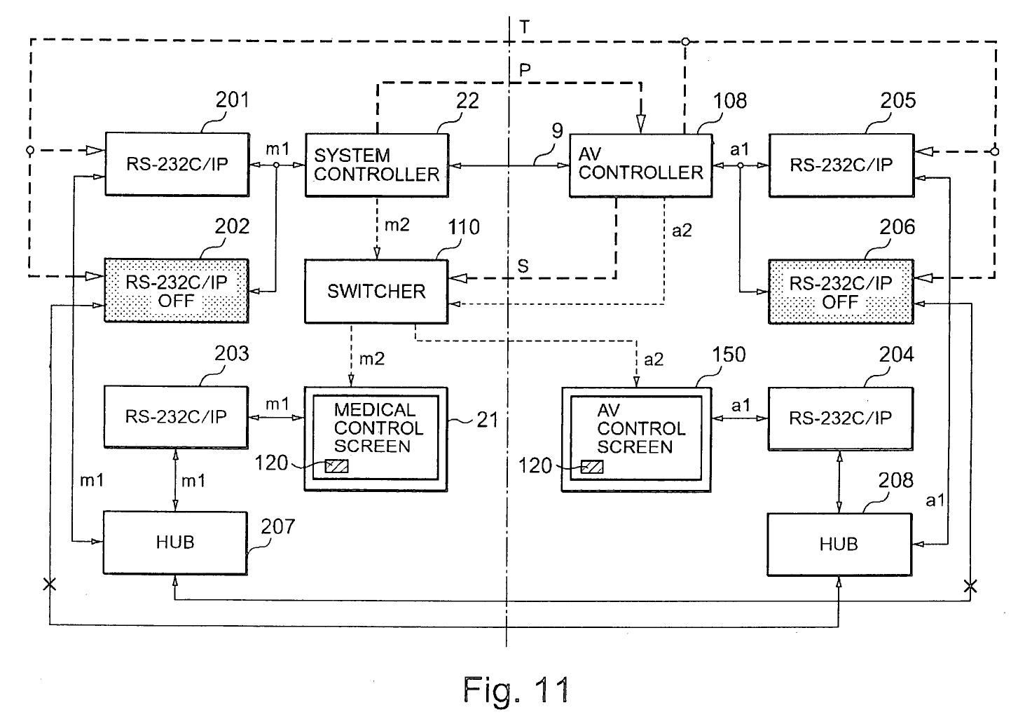

More specifically, as shown in FIG. 11, the switcher 110 outputs the video signal m2 from the system controller 22 to the operating panel 21 and outputs the video signal a2 from the AV controller 108 to the operating panel 150 in the initial state. This causes the switcher 110 to display a medical control screen on the operating panel 21 and to display an AV control screen on the operating panel 150.

Regarding the serial signals m1 and a1 in this state, the AV controller 108 turns off the RS-232C/IP 202 and the RS-232C/IP 206 by a control signal T.

Thereby, the information carried by the serial signal m1 from the system controller 22 is output to the operating panel 21, passing through the RS-232C/IP 201, the hub 207, and the RS-232C/IP 203 in this order. Similarly, the information carried by the serial signal a1 from the AV controller 108 is output to the operating panel 150, passing through the RS-232C/IP 205, the hub 208, and the RS/232C/IP 204 in this order.

Therefore, similarly to the second embodiment, the endoscopic surgery system 3 is controllable on the operating panel 21 and the AV system 100 is controllable on the operating panel 150.

Moreover, if an operator selects the switching button 120 displayed on the operating panel 21 using the touch panel function under the control of the endoscopic surgery system 3 with the operating panel 21, the selection signal is transmitted to the system controller 22 by means of the serial signal m1 and the system controller 22 transmits the information to the AV controller 108 by means of the parallel signal P.

Upon receiving the information on the switching signal, the AV controller 108 outputs a control signal S for switching of the switcher 110 and turns on the RS-212C/IP 202 and the RS-232C/IP 206 and turns off the RS-233C/IP 201 and the RS-232C/IP 205 by a control signal T.

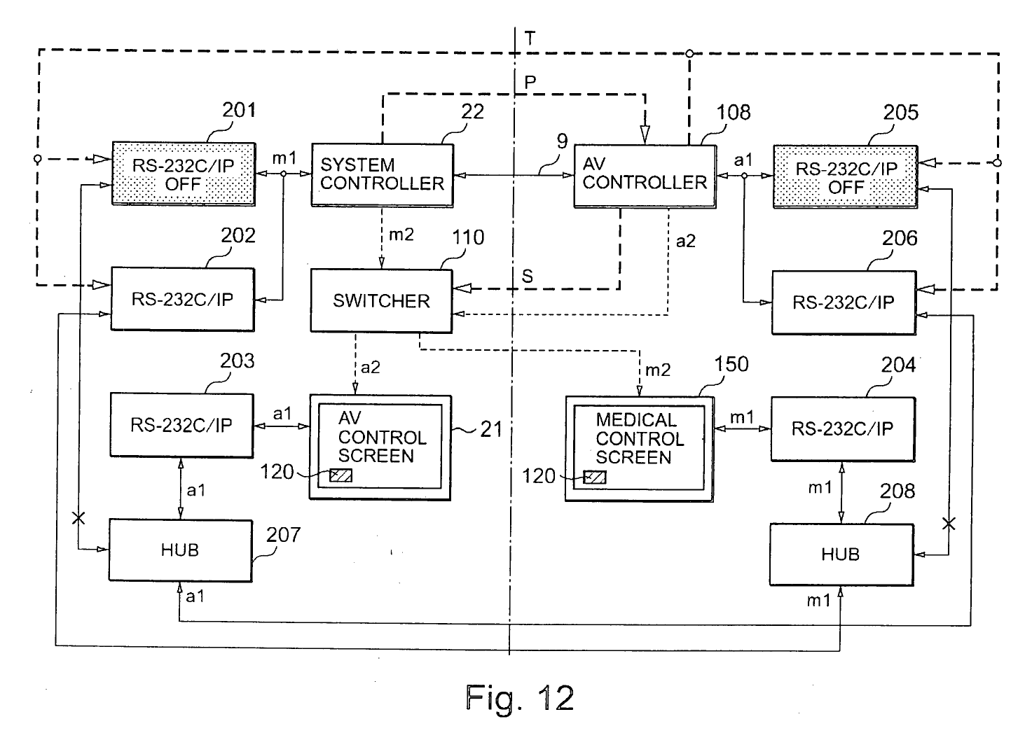

As shown in FIG. 12, the switcher 110 outputs the video signal m2 from the system controller 22 to the operating panel 150 and outputs the video signal a2 from the AV controller 108 to the operating panel 21 by the control signal S. Thereby, the switcher 110 displays a medical control screen on the operating panel 150 and displays an AV control screen on the operating panel 21.

On the other hand, the information carried by the serial signal m1 from the system controller 22 is output the operating panel 150, passing through the RS-232C/IP 202, the hub 208, and the RS-232C/IP 204 in this order by means of the ON/OFF control of the control signal T. Similarly, the information carried by the serial signal a1 from the AV controller 108 is output to the operating panel 21, passing through the RS-232C/IP 206, the hub 207, and the RS-232C/IP 203 in this order.

Therefore, similarly to the second embodiment, the endoscopic surgery system 3 is controllable on the operating panel 150 and the AV system 100 is controllable on the operating panel 21.

If the operator selects the switching button 120 displayed on the operating panel 21 using the touch panel function under the control of the AV system 100 with the operating panel 21, the selection signal is transmitted to the AV controller 108 by means of the serial signal a1, too. The AV controller 108 then outputs the control signals S and T, whereby the control state shown in FIG. 11 is resumed.

As stated hereinabove, in this embodiment it is possible to achieve the same effects as those of the second embodiment.

While there has been shown and described what are considered to be preferred embodiments of the invention, it will, of course, be understood that various modifications and changes in form or detail could readily be made without departing from the spirit of the invention. It is therefore intended that the invention not be limited to the exact forms described and illustrated, but constructed to cover all modifications that may fall within the scope of the appended claims.

1. An operating room control system comprising:
a medical device control system for controlling one or more medical devices for conducting a surgical procedure;
a medical support device control system for controlling one or more medical support devices, which support the surgical procedure;
an operating section for at least inputting a command to the medical device control system; and
an operating environment changing section for changing an operating environment of the operating section so as to enable at least input of the command to the medical support device control system from the operating section.

2. The operating room control system according to claim 1, wherein:
the operating section comprises a touch panel; and
the operating environment changing section selects between a display function and a sensing function of the touch panel of the operating section in synchronization with each other.

3. The operating room control system according to claim 1, further comprising a second operating section for at least inputting a command to the medical support device control system,
wherein the operating environment changing section selects between the operating environment of the operating section and an operating environment of the second operating section.

4. An operating room control system comprising:
a system controller for controlling one or more medical devices in a sterilized area;
an AV controller for controlling one or more medical support devices in a nonsterilized area;
an input unit for at least inputting a command to the system controller and the AV controller; and
a switcher for selecting between a signal path for connecting the system controller to the input unit and a signal path for connecting the AV controller to the input unit.

5. The operating room control system according to claim 4, wherein:
the input unit comprises a touch panel having a display function;
wherein if the signal path for connecting the system controller to the input unit is selected, information from the system controller is displayed on the touch panel; and
if the signal path for connecting the AV controller to the input unit is selected, information from the AV controller is displayed on the touch panel.

6. The operating room control system according to claim 4, wherein the switcher selects the signal path according to a signal from the input unit.

7. The operating room control system according to claim 6, wherein the signal is output in response to a predetermined operation from an operator using the input unit.
